(19) United States
(12) Patent Application Publication
(10) Pub. No.: US 2022/0061701 A1
(43) Pub. Date: Mar. 3, 2022

(54) SYSTEM AND METHOD FOR COMPRESSING AND SEGMENTING ACTIVITY DATA IN REAL-TIME

(71) Applicants: SRIDHAR KRISHNAN, RICHMOND HILL (CA); YASHODHAN ATHAVALE, MISSISSAUGA (CA)

(72) Inventors: SRIDHAR KRISHNAN, RICHMOND HILL (CA); YASHODHAN ATHAVALE, MISSISSAUGA (CA)

(21) Appl. No.: 17/312,799

(22) PCT Filed: Jun. 28, 2019
(86) PCT No.: PCT/CA2019/050909

§ 371 Date: Jun. 10, 2021

Related U.S. Application Data
(60) Provisional application No. 62/777,543, filed on Dec. 10, 2018.

(51) Int. Cl. A61B 5/11; G06F 3/0481; A61B 5/00; G16H 40/67; G16H 20/30; G01P 15/18; H03M 7/30

(52) U.S. Cl. CPC A61B 5/1118; G06F 3/0481; A61B 5/681; A61B 5/7232; A61B 2562/0219; G16H 40/67; G16H 20/30; G01P 15/18; H03M 7/30; A61B 5/7239

(57) ABSTRACT

There is provided a system, method and device for dynamically compressing an actigraphy signal at a source device. The method comprises receiving the actigraphy signal related to a user's physical activity from an accelerometer sensor on the source device and for compressing the actigraphy signal by determining regions of interest in the actigraphy signal to capture, said compressing performed by: computing a rapid change factor value indicating a drastic change in movement activity in said actigraphy signal, said rapid change factor computed based on determining a spurious free dynamic range of a second order difference signal of the actigraphy signal and subsequently determining the step size of the actigraphy signal, the step size indicating the interval with which the actigraphy signal instantaneously changes its value from one sample to another; automatically scanning the second order difference signal to locate samples in the second order difference signal having a value greater than the rapid change factor value, said located samples defining primary segment boundaries; -extracting frames of the encoded actigraphy signal between two consecutive primary segment boundaries and discarding outlying regions of the encoded actigraphy signal; and —outputting only the extracted frames representing a compressed actigraphy signal to an external computing device for subsequent processing.

### TECHNICAL FIELD

The present disclosure generally relates to methods and systems for compressing and segmenting real-time data and more particularly, long-term and real-time actigraphy data collected from actigraphs and other accelerometer based wearables (e.g. smart wearable devices) which monitor human activity and movement and are typically low-powered computing devices.

### BACKGROUND

Transmitting and/or storing data in low-power computing devices, such as wearables, requires a significant amount of power and memory, and this is an issue in such types of devices as they are limited by size and cost amongst other factors. The development of smart wearable devices within the IoT (Internet of Things) framework, has promoted the use of miniature sensors such as accelerometers, gyroscopes, and magnetometers to capture large amounts of sensor data from the human body to monitor daily activity. In one example, wearable devices use an accelerometer to detect physical activity. An accelerometer sensor provides three-dimensional coordinates that measure the acceleration force. Typically, accelerometer-based wearables, such as actigraphs, generate signals within the sampling rate of 16-2000 Hz with a resolution of 8-16 bits/sample. Currently available accelerometer-based wearables such as smartwatches generally over-quantize (i.e. quantize motion data more than necessary) and tend to sample the accelerometer sensor's signal infrequently. Such use of a computing device results in high memory and power usage when the data recording is intended for long-term monitoring of human activity (for example, 24-hour monitoring of daily activity).

Although state-of-art smart wearables fit aptly into the IoT framework, their feasibility for applications in long-term monitoring demands low power and memory consumption, reduced bit-rate, and promote an edge computing approach (i.e., process signals at the source to avoid computational overhead in cloud-based services). For wearable devices, despite their capacity to provide long-term recording and good battery life, for activity monitoring and recording operations, the processes of data acquisition, de-noising and analysis use conventional methods, which as noted above typically require significant power/memory usage and are not feasible and suitable for low-powered computing devices such as within the IoMT (Internet of Medical Things).

For example, existing methods of processing accelerometer data utilize conventional signal sampling, quantization and filtering techniques to pre-process data. Further, these conventional methods have focused on extracting generic statistical parameters without regards to the nature of the signal that is acquired and/or monitored and therefore are inefficient.

Accordingly, there remains a need for methods and systems for compressing and segmenting real-time actigraph data to address one or more of the above-mentioned deficiencies such as to improve signal acquisition, conserve battery and/or memory and enable long-term data recording.

### p SUMMARY

In one aspect, there is provided, a smart wearable device, the device comprising: (a) one or more sensors, wherein at least one sensor is an accelerometer configured to acquire an actigraphy signal indicating movement information related to a user; (b) a memory; (c) one or more communications interfaces; (d) a processor coupled to the memory; and (e) programming residing in a non-transitory computer readable medium, wherein the programming is executable by the computer processor and configured to: (i) receive input from the accelerometer providing the actigraphy signal; (ii) encode the actigraphy signal using an m-bit encoder; (iii) determine a first order difference signal of the encoded signal by subtracting every signal component from a value adjacent to it in the encoded actigraphy signal; (iv) determine a second order difference signal from the first order difference signal, by performing the operation in step (iii) again; (v) calculate a rapid change factor RCF using the second order difference signal, the rapid change factor RCF based on a spurious free dynamic range R of the second order difference signal, the step size SS

$$( SS = \frac{R}{{2}^{m} - 1} )$$

of the encoder such that the rapid change factor is calculated as

$$RCF\_factor = \frac{SS}{m \times {t}_{s}}$$

such that tₛ the sampling period of the signal; (vi) automatically define, in real-time, primary segment boundaries on the second order difference signal by: —scanning through the second order difference signal and determining signal samples whose value is greater than the RCF factor, wherein time values of the signal samples greater than the RCF factor define primary segment boundaries; (vii) extracting frames of the actigraphy signal which are between two consecutive primary segment boundaries, the extracted frames defining a compressed actigraphy signal for subsequent communication via the communication interfaces and discarding outlying regions of the second order difference signal.

In another aspect, there is provided a computer system comprising a wearable computing device communicating compressed actigraphy data to an external computing device: the wearable computing device comprising: 13 a computer readable memory; —an accelerometer sensor; —a processor coupled to the memory; —an application stored on the computer readable memory for execution by the processor for receiving an actigraphy signal related to a user's physical activity from the accelerometer sensor and for compressing the actigraphy signal by determining regions of interest in the actigraphy signal to capture, the compressing performed by: —computing a rapid change factor value indicating a drastic change in movement activity in the actigraphy signal, the rapid change factor computed based on determining a spurious free dynamic range of a second order difference signal of the actigraphy signal and subsequently determining the step size of the actigraphy signal, the step size indicating the interval with which the actigraphy signal instantaneously changes its value from one sample to another; —automatically scanning the second order difference signal to locate samples in the second order difference signal having a value greater than the rapid change factor value, the located samples defining primary segment boundaries; —extracting frames of the actigraphy signal between two consecutive primary segment boundaries and discarding outlying regions of the second order difference signal; and —outputting only the extracted frames to the external computing device.

In another aspect, there is provided a computer program product for dynamically compressing an actigraphy signal at a source device, the computer program product comprising a non-transitory computer-readable medium having computer readable code embodied therein executable by a processor for performing a method for compressing the actigraphy signal, the method comprising: —receiving the actigraphy signal related to a user's physical activity from an accelerometer sensor on the source device and for compressing the actigraphy signal by determining regions of interest in the actigraphy signal to capture, the compressing performed by: —computing a rapid change factor value indicating a drastic change in movement activity in the actigraphy signal, the rapid change factor computed based on determining a spurious free dynamic range of a second order difference signal of the actigraphy signal and subsequently determining the step size of the actigraphy signal, the step size indicating the interval with which the actigraphy signal instantaneously changes its value from one sample to another; —automatically scanning the second order difference signal to locate samples in the second order difference signal having a value greater than the rapid change factor value, the located samples defining primary segment boundaries; —extracting frames of the actigraphy signal between two consecutive primary segment boundaries and discarding outlying regions of the second order difference signal; and —outputting only the extracted frames representing a compressed actigraphy signal to an external computing device.

In another aspect, there is provided a method of determining regions of interest to extract in an actigraphy signal acquired by a wearable smart device, the method executed in a processor of the wearable smart device and comprising the steps of: a) computing a rapid change factor value indicating a drastic change in movement activity in the actigraphy signal, the rapid change factor computed based on determining a spurious free dynamic range of a second order difference signal of the actigraphy signal and subsequently determining the step size of the actigraphy signal, the step size indicating the interval with which the actigraphy signal instantaneously changes its value from one sample to another; b) automatically scanning the second order difference signal to locate samples in the second order difference signal having a value greater than the rapid change factor value, the located samples defining primary segment boundaries; c) extracting frames of the actigraphy signal between two consecutive primary segment boundaries and discarding outlying regions of the second order difference signal; and d) outputting only the extracted frames representing a compressed actigraphy signal to an external computing device.

### BRIEF DESCRIPTION OF THE DRAWINGS

In the accompanying drawings, non-limiting embodiments of the disclosure are described in detail below, with reference to the following drawings:

FIG. 1 is a block diagram of a network environment of a system for dynamically segmenting actigraphy data in real-time by a source device (e.g. a wearable smart device), in accordance with an embodiment;

FIG. 2 is a block diagram of a method for providing encoding for actigraphy signals in real-time according to the encoding module of FIG. 1, in accordance with an embodiment;

FIG. 3 is a block diagram of a method for providing segmentation of actigraphy signals in real-time according to the segmentation module of FIG. 1, in accordance with an embodiment;

FIG. 4 is a block diagram illustrating the segmentation module of FIG. 1 in further detail, in accordance with an embodiment;

FIG. 5 is a block diagram illustrating further components of the smart wearable device of FIG. 1 (e.g. a smart watch computing device), comprising the instructions module of FIG. 1 and communicating across the network of FIG. 1 with a server, in accordance with an embodiment;

FIGS. 6-9 are graphs illustrating various stages of an actigraphy signal as it is processed by selected components of the systems and methods of FIG. 1-5, in accordance with an embodiment;

FIG. 10 is an example schematic illustration of the operation of the system of FIG. 1, in accordance with an embodiment;

FIG. 11 illustrates various output graphs reflecting the operation of various stages of the system of FIG. 1, in accordance with an embodiment;

FIG. 12 illustrates a comparison between fixed and adaptive segmentation of actigraphy signals, in accordance with an embodiment;

FIG. 13 illustrates a schematic overview of the instructions module of FIG. 1, in accordance with an embodiment;

FIG. 14(a) illustrates graphs of an example tri-axial accelerometer signal shown along each axis and FIG. 14(b) illustrates a graph of a single axial accelerometer signal.

### DETAILED DESCRIPTION OF THE DRAWINGS

In one aspect of the disclosure, there is provided a system and method that is configured for extracting real-time relevant data from actigraphy signal(s). Generally, actigraphs measure human body displacement in single or tri-axial directions and are used in calculating gross motor activity for different applications. Typically, actigraphs are miniature devices which record and store motion data (e.g. smart watch) and can be used for fitness monitoring, calorie consumption, sleep/wake activity analysis and for rehabilitation therapies. That is, smart wearables embedded with accelerometers are typically termed as actigraphs in the art and the wearables shown in the enclosed figures according to embodiments of the disclosure are envisaged to include actigraphs or other wearable computing devices (including for example wearable electronic textiles, smart clothing, smart fabrics, wearable smart electronic rings, and other types of wearable smart electronics) having accelerometers and/or sensors (e.g. actimetry sensor) for collecting and processing real-time physiological data (e.g. movement/activity data).

The present disclosure discloses system(s) and method(s) to compress and segment actigraphy signals in real-time such as to capture and segment regions of interest thereby providing improved battery usage and memory consumption to store long-duration actigraphy data. First, the disclosed system and method enables less battery consumption while transmitting the continuous signal by encoding the continuous signal to a lower bit-rate. For example, it reduces quantization resolution to the lowest level possible without losing movement information. Second, the disclosed compressing and segmenting system and method improves memory conservation by compressing the continuous real-time signal to regions of interest (e.g. regions of activity). Additionally, the method and system disclosed preferably de-noises (e.g. to remove insignificant artifacts), segments and compresses the actigraphy data at the source device (e.g. the smartwatch or other smart wearable device) such that less information needs to be communicated across the network to another device (e.g. a server) for further analysis of the activity data, thereby addressing a computer related problem of bandwidth efficiency. Further, the compressing and segmenting system and method disclosed reserves battery and memory by capturing regions of interest through segmenting the continuous signal into active and inactive regions and storing the active continuous signal only. In one aspect, the continuous signal is a human physical activity signal. The active segment of the actigraphy signal is where there is at least a pre-defined amount of physical activity (e.g. brushing teeth, running, jogging, walking . . . ), and the inactive segment of the actigraphy signal is where there is no (or limited) physical activity (e.g. sitting or sleeping).

In an example implementation, when the compressing and segmenting system and method is used on a wearable smart device (e.g. an actigraph wearable), the encoding technique in the compressing and segmenting system has achieved a significant bit-rate reduction of 50-80%, a signal-to-noise ratio improvement of 20-90%, and a compression ratio of 50-90%.

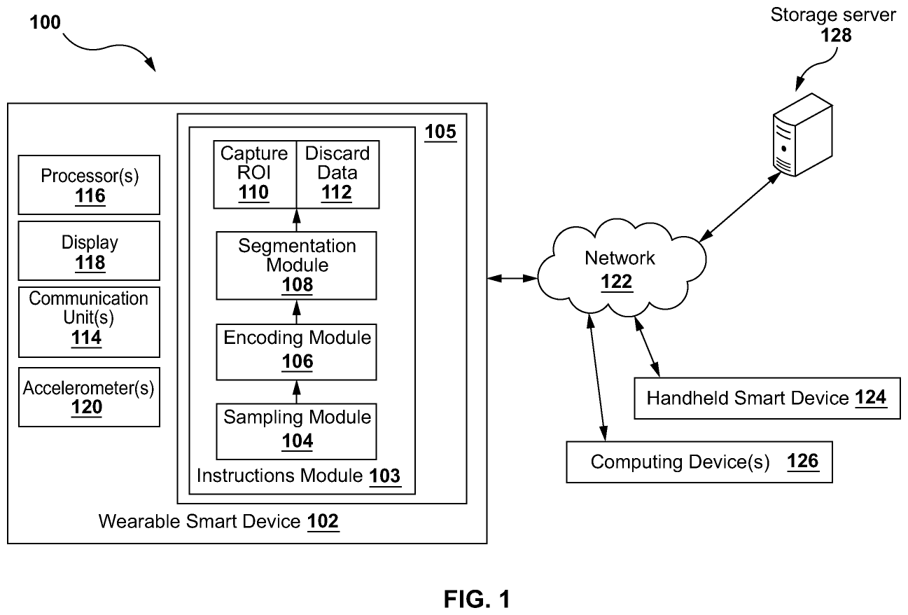

Referring to FIG. 1, shown is a schematic block diagram of an exemplary network environment 100 in which a system for communications with one or more wearable smart device(s) 102 are implemented. As noted above, the smart device(s) 102 can include smart electronic wearables such as smart watches, actigraphs, electronic textiles such as smart electronic clothing, smart electronic fabrics which include accelerometers or other sensors for capturing activity data. The network environment 100 is configured for processing and communicating data acquired by the wearable smart device 102, such as activity data captured by an accelerometer (e.g. 120) for further analysis and review. The network environment 100 comprises the wearable smart device(s) 102 (with communication unit(s) 114, processor(s) 116, display 118, accelerometer 120, instructions module 103 stored on memory 105). The instructions module 103 further comprises a sampling module 104, an encoding module 106, a segmentation module 108 for processing the activity data captured by the accelerometer 120 and outputting Region(s) of Interest (ROI) data 110 from the accelerometer data (e.g. capturing regions of activity) and discard data 112 (e.g. relating to regions of inactivity). The wearable smart device 102 communicates across a communication network 122 to other computing devices such as handheld smart device(s) 124, computing device(s) 126, and server(s) 128 including cloud storage servers for storing data relating to the connected computing devices. The communication network 122 may be a wide area network (WAN), a public network (e.g. the Internet) or a private network or a combination of same. Any of the communications between components 102, 124, 126, 128 may be via wired or wireless means. These computing devices typically communicate electronically using wired or radio (wireless) components using well-known protocols (e.g. Internet Protocols (IP). Further, the wearable smart device 102 may be configured to communicate with one or more other wearable smart devices via the network 122 or direct wired or wireless connection.

In at least one embodiment, the present disclosure provides systems and methods for optimizing memory and battery usage for a wearable device (e.g. 102). In one embodiment, the disclosed systems and methods address this issue by automatically and in real-time de-noising, compressing and adaptively segmenting of actigraphy data at the source (e.g. 102). That is, unlike other physiological signals such as ECG, actigraphy data visually appears to be spiky and transient in nature. Since it does not have any specific structure and only registers the amplitude of vibrations captured by the accelerometer it appears as randomly occurring values, exhibiting a non-normal nature such that characterizing and compressing the actigraphy data can be difficult. As illustrated in FIG. 1, the disclosed systems and methods utilize a lower-level quantization and provide an adaptive segmentation technique (e.g. adaptive to the user and/or the type of activity) as provided by software instructions module 103 on the wearable smart device 102. Notably, analysis and compression of actigraphy data provided by the accelerometer 120 occurs at the source device 102 (e.g. the acquisition devices) which captures/records the user's motion activity data, such as via accelerometer 120.

Referring to FIG. 1, the wearable smart device 102 provides compressing and segmentation via one or more processors or microprocessors 116 (e.g. such as a CPU), the processor(s) 116 performs computer processing operations and control operations necessary to execute software instructions (e.g. the instructions module 103) stored in an internal memory 105, such as one or both of random access memory (RAM) and read only memory (ROM), and possibly additional memory (not shown). The additional memory may comprise, for example, mass memory storage, hard disk drives, optical disk drives (including CD and DVD drives), magnetic disk drives (including LTO, DLT, DAT and DCC), flash drives, removable memory chips such as EPROM or PROM, emerging storage media, or similar storage media as known in the art and others for use with wearable smart devices. This additional memory (not shown) may be physically internal to the wearable smart device 102 or external. The processor 116 may retrieve items, such as applications and data lists stored on the internal memory 105 and/or on the additional memory to the internal memory, so that they may be executed or to perform operations on them. The communication unit(s) 114 further provide an interface (e.g. a network interface) that allows software and data to be transferred between the components of the device 102 and/or externally to external systems and networks, such as computing device(s) 126, handheld smart device 124, and server 128. Software and data transferred via the communication unit(s) 114 can be in the form of signals which can be electronic, acoustic, electromagnetic, optical or other signals capable of being received by the communication unit(s) 114.

Wearable smart device 102 is typically capable of, for example, monitoring one or more biological or physiological characteristic of a user wearing the device via input received from one or more sensors on the device 102, including monitoring movement and physical activity via the accelerometer 120.

As will be understood by a person skilled in the art, wearable smart device 102, may include any wearable device such as a smart watch. As further understood, wearable smart device 102 may have some components and functions in common with handheld mobile device 124, however as envisaged herein, the segmentation and compression operations of the accelerometer data occur on the source device (e.g. the wearable smart device 102) such as to optimize memory usage on the device 102 and minimize the amount of activity data to be transferred. Specific examples of wearable smart devices may include Pebble™ smart watch, Fitbit™, Apple™ watch, Garmin™, Sony™ smart watch or the like but can comprise other smart devices (e.g. smart electronic rings) as understood by a skilled person.

Computing device 126 may be a personal computer, a laptop computer, a tablet, a mobile device or other electronic device with applications and software to communicate with wearable smart device 102 and obtain actigraphy data therefrom for further display, analysis and review (e.g. via a user interface).

Similarly, handheld smart device 124 is a mobile computing device configured to communicate with device 102 and receive actigraphy data therefrom.

Devices 124 and 126 and server 128 may be further configured to receive user input (e.g. via a user interface) to provide additional indications that a user of the wearable smart device 102 is active/inactive (e.g. manual user input indicating activity and activity time/duration) such that the processor 116 is further configured via the instructions module 103 to analyze said indications and provide said indications to the segmentations module 108 for improved machine learning and to better define the segmentation boundaries in the module 108 for providing output data 110 and 112.

FIG. 1 illustrates a functional block diagram of the network environment 100 including a wearable smart device 102 configured, via the instructions module 103, which when executed by the processor 116 configure the device 12 for reading, compressing and segmenting long-term real-time actigraphy signals (e.g. as obtained from accelerometer 120) to output only regions of interest depicting regions of physical activity in the form of captured region of interest (ROI) data 110 to external devices such as cloud services on server 128. Sampling module 104 continuously samples an incoming signal (e.g. as received from accelerometer 120). The encoding module 106 is configured to encode the signal to improve signal-to-noise ratio and compress the data. The segmentation module 108 is further configured to receive the encoded data from the encoding module 106 and segment the encoded data to capture regions of interest 110 within the encoded accelerometer data (e.g. regions of high activity) and discard data 112 that correspond to regions without useful information related to activity/movement of the user. The data corresponding to regions of interest 110 are transmitted via network 122 for subsequent use (e.g. storage on server 128 or further analysis by handheld smart device 124/computing device 126).

Generally, in one embodiment as illustrated in FIG. 1, the instructions module 103 is configured to provide signal segmentation and compression at the source (e.g. on the wearable smart device 102) which acquires the actigraphy signal such as to eliminate the need to digitize the actigraphy signal first and then encode it. The encoding module 106 is configured to encode the sampled data as received from the sampling module 104 directly into a low-level bit resolution such as to preferably avoid memory leakage and increase computational efficiency.

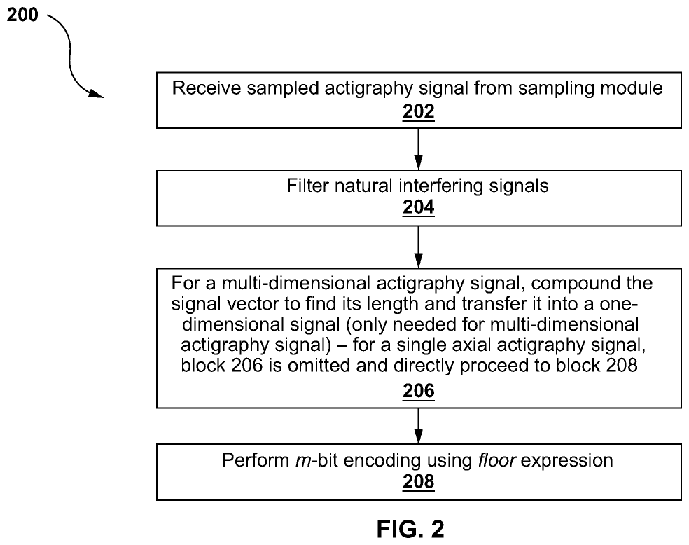

Referring to FIG. 2, shown is an example flowchart illustrating the method 200 performed by the encoding module 106 of FIG. 1 (e.g. when executed by the processor 116) for actigraphy signal filtering and compression. The encoding module 106 is configured to pre-process, condition and smooth the actigraphy signal prior to segmentation by the segmentation module 108 based on activity analysis. Thus, the encoding module 106 pre-processes and de-noises the continuous actigraphy signal (e.g. as provided by the accelerometer 120 and sampled by the sampling module 104). The de-noising process may include for example, removing insignificant signals or portions of the signal from the actigraphy signal. This m-bit signal encoding method reduces memory consumption by compressing the signal and increasing its quality. Hence, it enables faster and more efficient signal transmission of the actigraphy signal for subsequent analysis. In this example, at block 202, the encoding module 106 receives, from the sampling module 104, tri-axial (three-dimensional) accelerometer signal(s) S={x, y, z}ₙ where x is acceleration, y is intensity, z is direction or angle of movement, n is the time unit sample. Alternatively, the encoding module 106 receives a single-axial actigraphy signal at block 202 for encoding. At block 204, the encoding module 106 then normalizes the received signal using pre-defined (e.g. manufacturer's specifications) to reduce the effects of the earth's gravitational force on the actigraphy signal such as to enhance sample amplitudes without losing movement information. Thus, at block 204, the earth's gravitational force normalization is performed using manufacturer's specific option to enable or disable the sensor to send a normalized signal. At block 206, the encoding module 106 then performs vector compounding by finding the length of the vector continuously and in real-time as V=\sqrt{x²+y²+z²}, for all n samples where n is the number of samples/values in the signal. Vector compounding combines the tri-axial activity for each recorded movement, thus ensuring that the effect of each axis is considered without information loss. As will be envisaged by a person skilled in the art, vector compounding is only applicable to tri-axial data and not to single axial data. Thus, block 206 is optional and not needed in the case of single-axial actigraphy data but rather used in the case of multi-axial data.

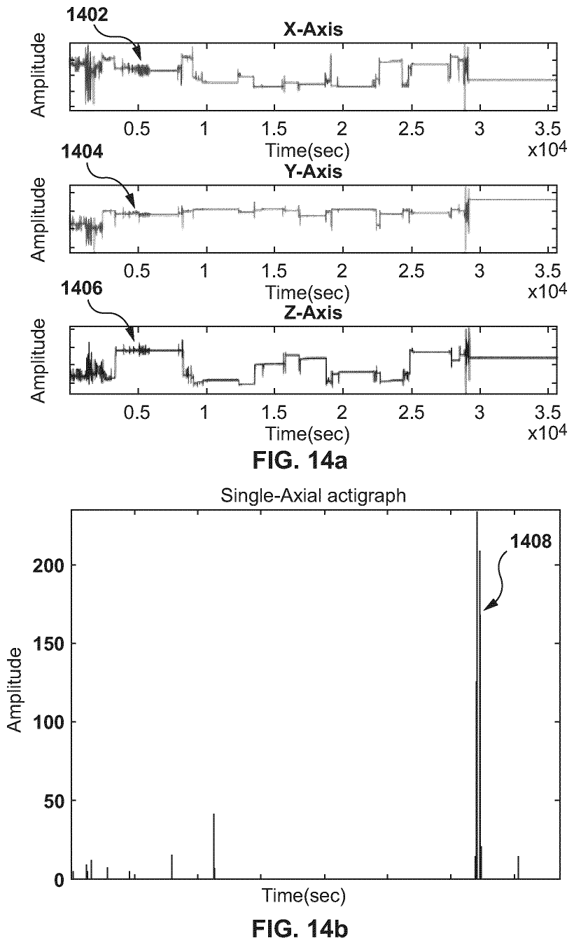

Generally, accelerometer 120 may be configured to capture physical movement of a user in tri-axial mode (x,y,z) such that each direction represents one of the three movement components, namely—acceleration, intensity and movement angle. See for example, FIG. 14(a) which depicts a tri-axial accelerometer signal along three different axis (1402, 1404 and 1406). Alternatively, accelerometer 120 may be a single axial accelerometer sensor, which records a single value per user's physical movement. See for example, FIG. 14(b) which depicts a single axial accelerometer signal 1408.

At block 208, the signal is then encoded using m-bit encoding to quantize the signal. The m-bit encoding at block 208 is achieved through two stages, first by finding the swing factor for m-bit encoding then introducing the signal to the floor formulation. This operation encodes the quantized signal to a lower bits/sample than the original sampled signal. The swing factor is signal independent, and it depends on the desired quantization level. In one example, m number is high, and this produces a high resolution and high memory consumption. In another example, the m is low, and this produces less resolution and less memory consumption. The swing factor is measured as

$$swing = \frac{{2}^{m} - 1}{2} ,$$

where m is the desired quantization level. In at least one aspect, the m-bit encoding is preferably a 3-bit encoder with m=3. That is, experimental results performed for the present disclosure show that the 3-bit encoding provides the highest signal clarity for an actigraphy signal. In one example, the linear discriminant analysis (LDA) classification accuracies of signals encoded using different bit-factors were compared for multiple dataset. It was found according to the present disclosure that 3-bit encoding of actigraphy data ensures highest performance in data acquisition, storage and analysis. Further to this, it was observed in accordance with the present disclosure, in experimental results that 3-bit encoding provides the highest activity recognition rate.

The encoded signal provided at block 208, Q, using the floor formulation, is Q=[V×swing+swing]. The floor formulation noted above at block 208 digitally approximates each value generated from Q=[V×swing+swing] to the greatest integer less than or equal to it. For example, a value of 3.4 would be mapped to 3. The m-bit encoding also acts as a de-noising step by removing the effect of acceleration due to gravity, vector compounding, and lower bit encoding, which further reduces the amplitude of noise components.

Typically, an accelerometer or actigraphy signal is digitized with a fixed number of bits/sample resolution as defined by the manufacturer specifications. The m-bit encoding at block 208 thus further encodes the signal into a lower bits/sample resolution, using a set of discrete integer values. For example, if the raw actigraphy signal was originally quantized with a r-bit resolution. In order to further compress it, denoise it and remove insignificant and redundant values, it is then encoded with an m-bit resolution at block 208, such that m<<r. This effectively reduces the bit rate of the actigraphy signal. Using a pre-defined swing factor, the encoding is performed using a floor operation, which maps each sample value in the raw actigraphy signal to the nearest integer greater than or equal to it. For example, if the value of the i^{th} component in the raw actigraphy signal Sᵣ(i)=5.4, then once 3-bit encoding is performed:

$$Q = [ S r ( i ) x \frac{{2}^{3} - 1}{2} + \frac{{2}^{3} - 1}{2} ] = [ 22.4 ] = 22 ,$$

such that an amplitude of 5.4 gets assigned a discrete value of 22 after the floor and encoding operations of block 208. In this way, the lower bit encoding, further reduces the amplitude of noise components.

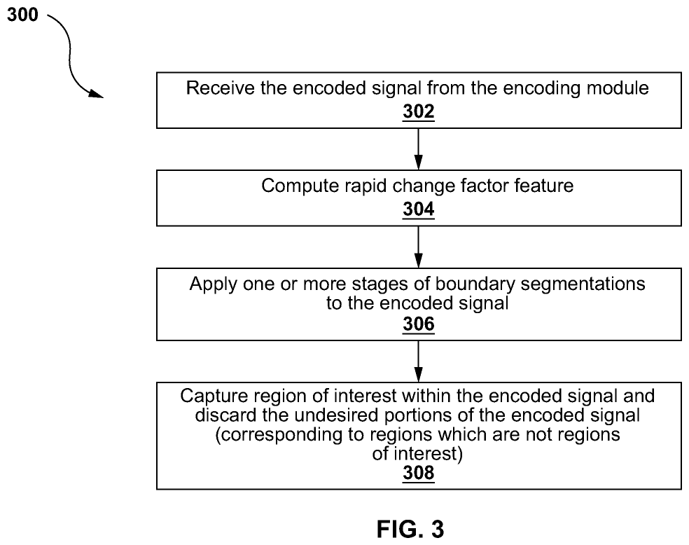

FIG. 3 is a flowchart illustrating an example operation of method steps 300 performed by the segmentation module 108 of FIG. 1, in accordance with an embodiment. After encoding the analog signal in the encoding module 106, the signal is segmented in the segmentation module 108 to capture regions of interest (e.g. regions of high movement/activity in the encoded actigraphy signal). Generally, the segmentation module 108 is configured to compute first and second order difference versions of the encoded signal obtained from encoding module 106 and by computing these difference versions, and thereby exploits the non-stationary nature of the physiological actigraphy signal such as to assess for regions where its second order statistics change.

At block 302, the segmentation module 108 receives the encoded signal from encoding module 106 and computes a rapid change factor feature (RCF) from the encoded signal at block 304. As will be described, in order to calculate the RCF, a first order difference of the encoded signal is computed followed by a second order difference of the encoded signal by the processor 116. The RCF is unique to each actigraphy signal (and unique to each user) and is used by the segmentation module 108 to detect events of interest from the spiky and transient continuous actigraphy signal (e.g. can detect when there's a drastic change in a signal and thus an indication of movement/activity).

The segmentation module 108 uses the RCF to detect a region of interest by locating the onset (e.g. start) and offset (e.g. end) of the region of interest in the actigraphy signal (e.g. region indicating high activity/movement of a user). In one aspect, the RCF can be applied to a second order difference of the actigraphy signal such as to scan the signal and determine a time instance when the second order difference of the actigraphy signal exceeds the RCF (this time instance can be the start of a first region of interest) and then continue scanning the signal until the second order difference signal drops below the RCF value (this time instance can be the end of the first region of interest). In yet further aspects, once the regions of interest are located, the duration of each region of interest may further be modified depending upon expected durations of time associated with pre-defined activity types. For example, if the segmentation module 108 receives indication that the user is performing a pre-defined type of activity (e.g. based on prior history of the user actigraphy signals) then a reference duration of time may be associated with that pre-defined type of activity and the regions of interest located by the RCF factor may further be modified by the reference duration of time. The segmentation module 108 then classifies each located region of interest with its respective activity type based on pre-defined templates (e.g. defined manually, automatically or semi-automatically such as based on prior history of similar activity). Stored on internal memory 105 of the device (or on an externally accessible memory) is stored a database of templates that corresponds to various activities. This database is used to generate a trained process that the segmentation module uses to classify the region of interest with its respective activity. RCF is unique for each person's activity, the activity tracked by a wearable smart device such as a smart watch or a smart phone.

To compute RCF at block 304, the segmentation module 108 determines whether the slope of the encoded signal received from block 302 is increasing or decreasing. Notably, the RCF parameter depicts the drastic rate of change in sample value for the entire second order difference signal. Therefore, RCF computation is based on the second order statistics of the continuous signal (encoded actigraphy signal). Thus, RCF calculation does not utilize a fixed threshold to operate. Rather, the segmentation module 108 updates the threshold based on history of the signal, e.g. past signal's samples encounters. The first stage of RCF computation at block 304 involves calculating first-order difference and second-order difference of the encoded analog signal representing the actigraphy signal. The first order difference, dQ=[Q₁, Q₂-Q₁, . . . , Qₙ-Qₙ₋₁] calculates the slope of the encoded analog signal. It is computed by subtracting the previous adjacent encoded sample from the sample value. Put another way, the first order difference signal is computed by subtracting every signal component from the value adjacent to it in the encoded signal. Subsequent to calculating the first order difference signal, the second difference signal is computed by the segmentation module 108 by performing the steps performed for the first order difference signal once again. Notably, second order difference is computed as 2dQ=[dQ₁, dQ₂-dQ₁, . . . , dQₙ-Qₙ₋₁] which calculates the slope of the slope to find if it is an increasing or decreasing slope, and n is the number of signal samples. As can be seen, the computation of the first and second order differences, further changes each sample value to a discrete integer.

After computing the second-order difference signal by the segmentation module 108, the spurious free dynamic range is calculated by segmentation module 108 at block 304. The spurious free dynamic range R of the encoded signal is the ratio of the fundamental component in the encoded signal to its highest peak value. It is calculated as:

$$R = s f d r ( 2 d Q , f s ) = 20 \times {log}_{10} \frac{RMS Amplitude of Fundamental Component}{RMS Amplitude of the Highest Peak Va1ue} \qquad ( 1 )$$

where R=Amplitude of Fundamental (dB)-Amplitude of largest spur (dB), fs is the sampling frequency and RMS is the root mean square of the continuous signal.

Put another way, to calculate the spurious free dynamic range (sfdr) shown as value R of the second order difference signal, the range R is the difference in decibels between the amplitude of the fundamental or RMS (root mean square) value of the signal, and the amplitude of the largest spur or peak in the signal. The range R can be computed using the following expression

$$R = \sqrt{\frac{1}{n} ( 2 d {Q}_{1}^{2} + 2 d {Q}_{2}^{2} + … 2 {dQ}_{n}^{2}} - {max}_{n} 2 d Q \qquad ( 1 a )$$

After calculating the spurious free dynamic range by the segmentation module 108, R, RCF computation requires step-size calculation of the signal encoder (e.g. encoding module 106). It is noted that the step size is different from the bit resolution of the signal. The step-size denotes the interval with which the signal instantaneously changes its value from one sample to another. Therefore the step-size is computed as:

$$SS = \frac{R}{{2}^{m} - 1} \qquad ( 2 )$$

Subsequently at block 304, using the spurious free dynamic range and the step size, the rapid change factor RCF is then calculated as:

$$RCF\_factor = \frac{SS}{m \times {t}_{s}} \qquad ( 3 )$$

where

$${t}_{s} = \frac{1}{{f}_{s}}$$

is the sampling period of the single and fₛ is the sampling frequency of the signal in as provided by the sampling module 104.

As discussed earlier, the RCF value will be unique for each type of activity captured and would also be unique to the particular individual for which an activity is recorded for.

After calculating RCF factor of the signal at block 304, then at block 306, a second order difference version of the signal preferably undergoes two stages of boundary segmentation by the segmentation module 108, e.g. finding primary segment boundaries and secondary segment boundaries. The first stage excludes any signal sample within the second order difference signal that has a second-order difference less than the RCF factor value, this stage finds the primary segment boundary. In one example, time-stamps are located. Time-stamps correspond to the duration of the region of interest. It is the duration between the two boundaries. For example, the second order difference signal is scanned to determine a time instance when the signal value is greater than the RCF value, this time instant is defined as the start time of a first region of interest (e.g. tₛₜₐᵣₜ) for the primary segment boundary. The second order difference signal is then scanned (subsequent to tₛₜₐᵣₜ) until the signal value drops below the RCF value, this time instant is defined as the end time of the first region of interest (e.g. t_{end}). This process repeats until all of the regions of interest are located within the second order difference signal. The second stage of boundary segmentation depends on the activity itself by finding the first-order difference of the first stage and compare it to the activity time reference (e.g. t_{ref}) . These boundaries are the secondary segment boundaries. That is, secondary or final segment boundaries are application or activity specific boundaries (e.g. templates of such boundary durations may be stored in memory 105 as shown in FIG. 1). For example, while climbing stairs, peak accelerometer activity occurs every 1 second. Therefore, to compute the secondary boundaries as: SSB=(t_{PSBi}-t_{PSBi+1})≥t_{ref} such that t_{ref} is the corresponding time between activity events.

In one example, boundaries other than the secondary segment boundaries are calculated. These boundaries are final segment boundaries. Final segment boundaries are decided when a specific number peaks surpasses the average amplitude of the entire continuous signal. In one example, the average amplitude of a user defined duration instead of the entire continuous signal.

At block 308, after the signal undergoes one or more stages of boundary segmentation in block 306, the boundary segments (e.g. primary segments or if applicable secondary segments or if applicable final segments) defined in block 306 are used such that segments within two defined consecutive segment boundaries are kept (e.g. output as captured regions 110) and the remaining segments are discarded (e.g. defined as discarded data 112 in FIG. 1).

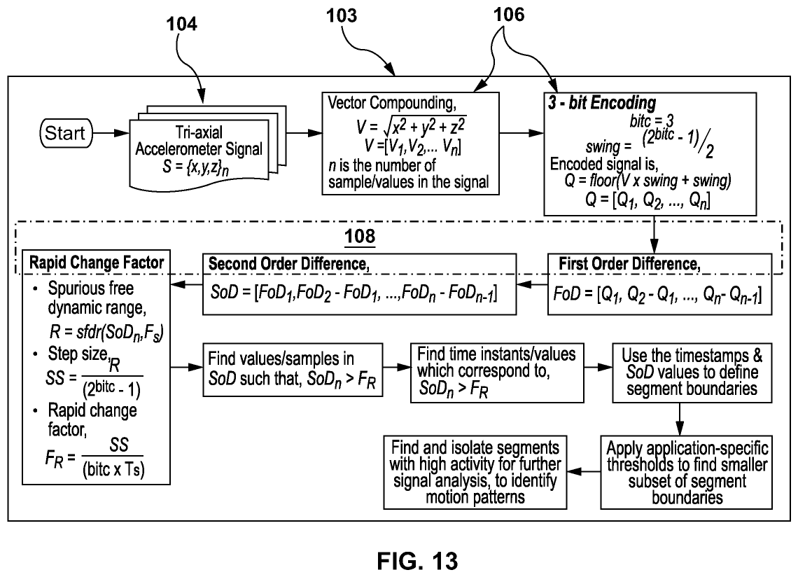

Referring now to FIG. 13 shown is an overview of the instructions of module 103 including module 104, 106 and 108 instructions for processing an actigraphy signal and providing a segmented output signal (e.g. a compressed signal) of desired regions of interest in the actigraphy signal.

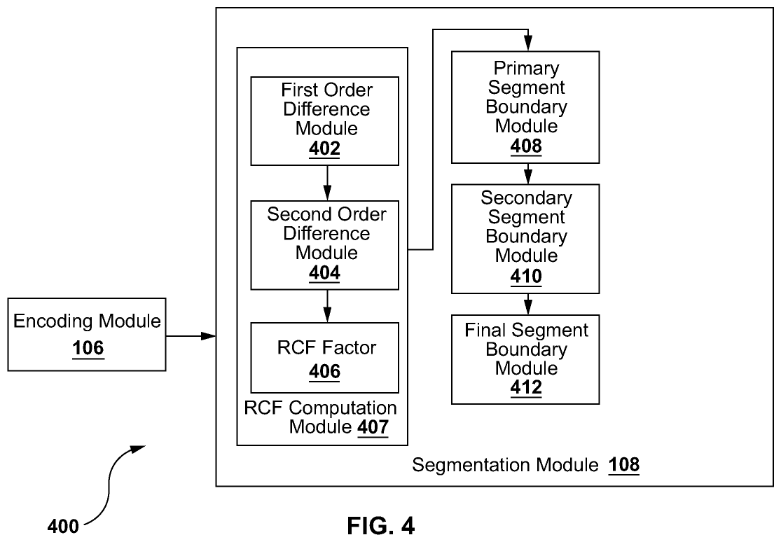

FIG. 4 illustrates a schematic diagram illustrating further details of the segmentation module 108 of FIG. 1 for segmenting an actigraphy signal from a wearable smart device, in accordance with an embodiment. Segmentation module 108 receives the m-bit encoded signal from the encoding module 106. The segmentation module 108 further comprises software modules RCF computation module 407 (including first order difference module 402, second order difference module 404, RCF factor module 406) and primary segment boundary module 408, secondary segment boundary module 410, final segment boundary module 412. The first and second order difference of the continuous signal obtained from the encoding module 106 is computed in the first order difference module 402 and second order difference module 404, respectively. These two stages exploit the non-stationary nature of the physiological actigraphy signal, and assess for regions where the signal's second order difference change. The first order difference module 402 calculates the value of every time sample by subtracting the past sample value from it. For example, if the first four-samples of the continuous signal from the encoding module has consecutive values Q=[5, 22, 10, 3], then its first order difference is dQ=[5, 22−5, 10−22,3−10]=[5, 17,−12, −7]. This is an example of a four-sample-output from a stream n samples from the first order module. Second order difference module 404 calculates the value of every time sample by subtracting the past first order difference value from it. Using the same example, 2dQ=[5,17−5, −12−17, −7−(−12)]=[5,12, −29,5].

RCF factor module 406 utilizes the value of 2dQ to calculate its value. The first stage in calculating RCF factor is by calculating the spurious free dynamic range, R, which is a single value for the signal, and it is calculated following equation (1). Step-size and RCF are calculated following equations (2) and (3), respectively. Consequently, the value for RCF factor is a single value that corresponds to the respective set of activities performed within the time segment where RCF was calculated on.

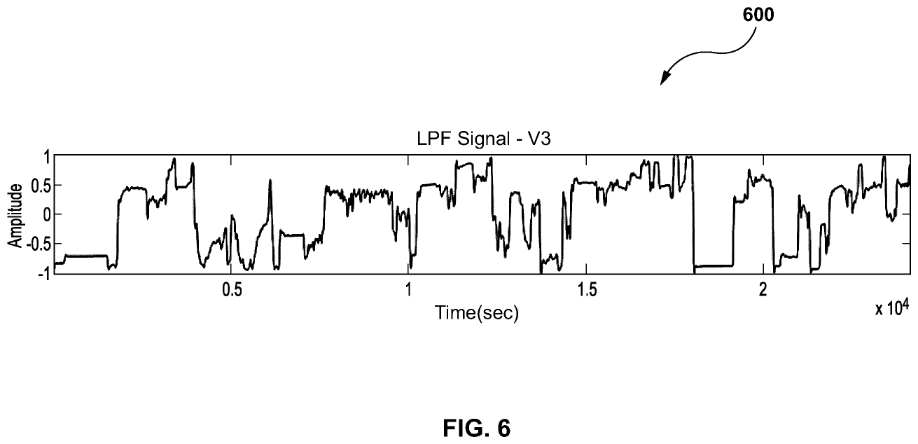

An example operation of defining boundary segmentation and capturing regions of interest (e.g. blocks 306 and 308 of FIG. 3) is further shown in FIGS. 6-9. FIG. 6 schematically illustrate an example second order difference signal 600 (e.g. as provided in module 404 of FIG. 4 and by segmentation module 108 of FIG. 1). Initially, the primary segment module 408 (see FIG. 4) locates samples and corresponding time stamps in the second order difference signal 2dQ shown in FIG. 6 such that, 2dQ>RCF. Notably, the primary segment boundaries (PSB) are located whenever there is a value where 2dQ is greater than the RCF factor value, the time-stamps for these events correspond to PSBs.

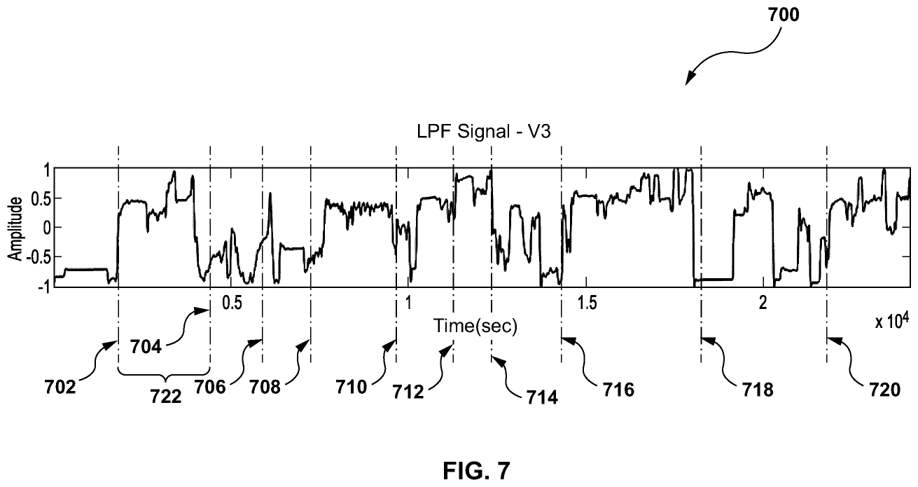

FIG. 7 illustrates a graph 700 indicating the primary boundaries located from FIG. 6. The primary segment boundaries are located by scanning through the entire second order difference signal of FIG. 6 and locating samples whose value is greater than the rapid change factor RCF. Corresponding to these samples, the segmentation module 108 of FIG. 1 and specifically the primary segment module 408 of FIG. 4 is configured to find the time-stamps from the second order difference signal. These time stamps are visually indicated in FIG. 7. In one embodiment, the segmentation module may perform only a primary segment boundary module operation such as to segment the signal after the primary segment boundary module 408 operation. In such embodiment, only those parts or frames of the actigraphy signal which are between two consecutive primary segment boundaries (see FIG. 7) are extracted and the remaining portions corresponding to outlying regions of the second order signal are discarded by the processor 116. As discussed above, while scanning through a second order difference signal of the actigraphy signal, a start time stamp (tₛₜₐᵣₜ)a first region of interest is defined when a , -start, of value of the second order signal sample exceeds the RCF value and the end time (t_{end}) of the first region of interest is the last time instant before a value of the second order signal sample drops below the RCF value. Thus, the start and end time stamps correspond to a duration of time when the second order difference signal exceeds the RCF value. This process is repeated until all of the start and end times for all regions of interests are determined. In at least one aspect, these time instances of the regions of interest (as determined by comparison of the second order difference signal to the RCF value) can then be applied to the original actigraphy signal (e.g. encoded actigraphy signal) to extract portions of the actigraphy signal within each region of interest (e.g. between tₛₜₐᵣₜ and t_{end}) for the primary segmentation boundaries.

Referring to FIG. 7, the boundaries 702, 704, 706, 708, 710, 712, 714, 716, 718, and 720 are the primary boundaries of the signal 700. An example of an activity is as shown in time duration or primary segment boundary 722, it is the time between two primary boundaries. Referring again to FIG. 4, the secondary boundaries module 410 further finds subset of segment boundaries. The secondary boundaries are application specific in nature, and their computation depends on the duration of the activity captured by the actigraph computing device. For example, if the activity to perform a task takes 1 second, then the region of interest corresponds to the regions where the duration between consecutive primary boundaries is greater than 1 second.

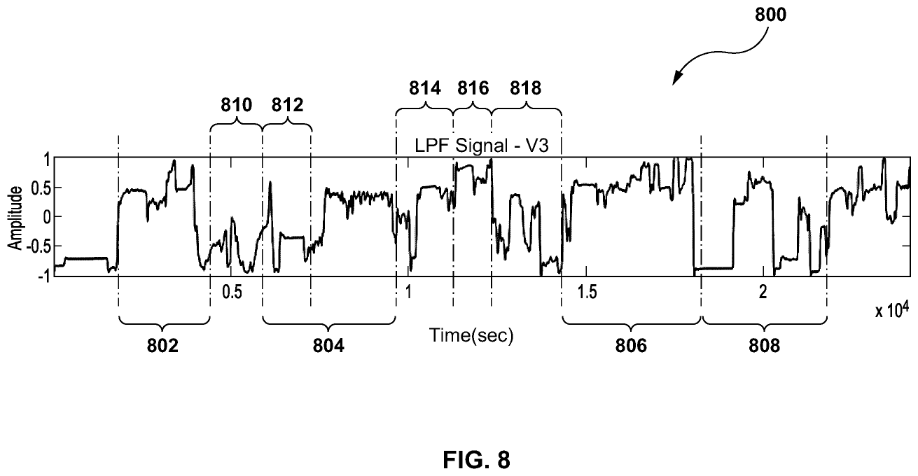

FIG. 8 illustrates a graph 800 of the secondary segment boundaries of the second order actigraphy signal as computed by the segmentation module 108 of FIG. 1 (or in the secondary segment boundary module 410 of FIG. 4). Each of the durations depicted by 802, 804, 806, and 808 has a difference between two consecutive primary boundaries greater than 1 second; hence, it is a region of interest. However, durations shown as 810, 812, 814, 816, and 818 are of less than 1 second hence they are not regions of interest, and they are discarded by the segmentation module 108 such that only regions of interest are output by the segmentation module 108.

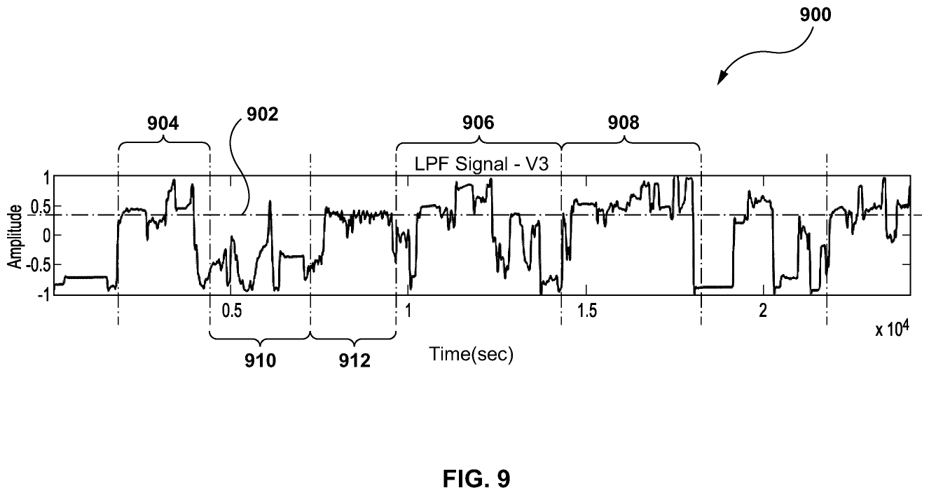

FIG. 9 illustrate an output example graph 900 of the final segment boundary module 412 of FIG. 4 within the segmentation module 108. Final segment boundary module 412 shown in the embodiment of FIG. 4 and is a final refinement for capturing region(s) of interest in the actigraphy signal, and this is an optional stage. It is understood that regions of interest produced in FIG. 8 are sufficient to extract regions of interest, in accordance with an embodiment. However, to further conserve memory, in one embodiment, this stage may be utilized. It is achieved by finding the average value of the amplitude of the entire signal 902. The final region corresponds to the secondary regions where there are a user specified number of amplitude higher than the average value as in durations 904, 906, and 908, and they are considered as regions of interest whereas regions 910 and 912 are discarded.

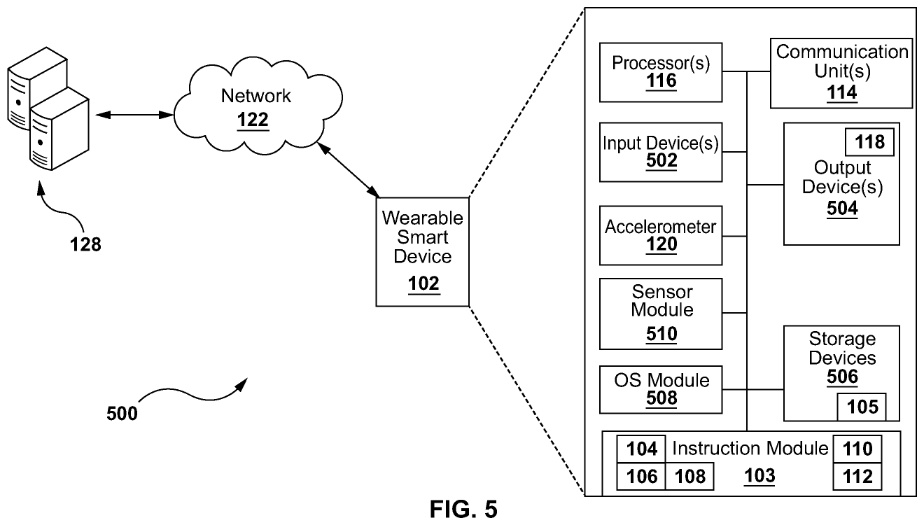

FIG. 5 depicts a simplified schematic block diagram of a system 500 for processing actigraphy data and further illustrates the computing components of the wearable smart device 102 of FIG. 1 as a computing device in communication with one or more computing devices such as storage server 128, handheld smart device 124, computing device(s) 126 across a network 122, in accordance with an embodiment. Further example components of wearable smart device 102 are shown schematically. Wearable smart device 102 comprises one or more processors 116, one or more input devices 502. Input devices 502 may be key pads, buttons, microphone or an optical input device, etc. Wearable smart device 102 comprises one or more output devices 504 which may include a display screen 118, a speaker, light, bell, vibratory device, etc. Device 102 also comprises one or more communication units 114 for communicating via one or more networks (e.g. network 122). The wearable smart device 102 further comprises one or more storage devices 506 including storage 105. The one or more storage devices 506 may store instructions and/or data for processing during operation of wearable smart device 102, including instruction module 103 and components 104, 106, 108, 110, 112. The one or more storage devices may take different forms and/or configurations, for example as short term memory or long term memory. Storage devices 506 including internal memory 105 store instructions and/or data for device 102, which instructions when executed by the one or more processors 116 configure the device 102. The instructions may be stored as sampling module 104, encoding module 106, segmentation module 108, and capture module 110 and discard data module 112.

Wearable smart device 102 also hosts an operating system 508. The communication between the modules is performed by bus/communication unit 114. Bus 114 may be a high-speed system interface or a high-speed peripheral interconnect bus, such as the PCI, PCI-express, or the like.

Referring to FIG. 5, the wearable smart device 102 further comprises a sensor module 510 that continuously monitors health and fitness statistics of a user and generates a signal based on monitoring real-time human activity and movement. The continuous signal is sampled by sampling module 104 with a sampling frequency less than the sensor module frequency. The compressing and segmenting system encodes and segments the sampled continuous signal. In one aspect, the continuous actigraphy signal generated from the compressing and segmenting system is stored locally in storage device 506. In another aspect, the continuous signal generated from the compressing and segmenting system is transferred via the network 122 to one or more other computing devices e.g. server 128, computing device 126, handheld device 124 (see also FIG. 1).

Computing device 102 can be a smartwatch, in other examples, it may be an actigraph, cell phone, tablet, tabletop computer, a laptop computer, etc. Other computing devices 128 in FIG. 5 may be servers or cloud services such as Microsoft Azure, Google Cloud, Amazon Web Services (AWS), etc.

FIG. 5 is one example of a computing device for obtaining actigraphy data such as wearable smart device 102. Wearable smart device 102 can be an actigraph with a computer application to detect daily physical activities, in one aspect. In another aspect of the actigraph (e.g. wearable smart device 102), it may be used for fitness purposes. In another aspect of the actigraph (e.g. wearable smart device 102), it may be used for long-term monitoring of individuals suffering from a neuromuscular disorder. In another aspect of the actigraph (e.g. wearable smart device 102), it may be used for daily activity for the general population.

In one aspect, the sensor module 510 includes an accelerometer 120 that measures acceleration and generates a three-dimensional continuous signal. This accelerometer 120 can be used in physical activity detection. In another aspect, the sensor module is a temperature sensor. In another aspect, it is a speed sensor or any other sensor that generates a continuous signal. In another aspect, the sensor module 510 may comprise gyroscopes. In another example, the sensor module 510 may comprise magnetometers.

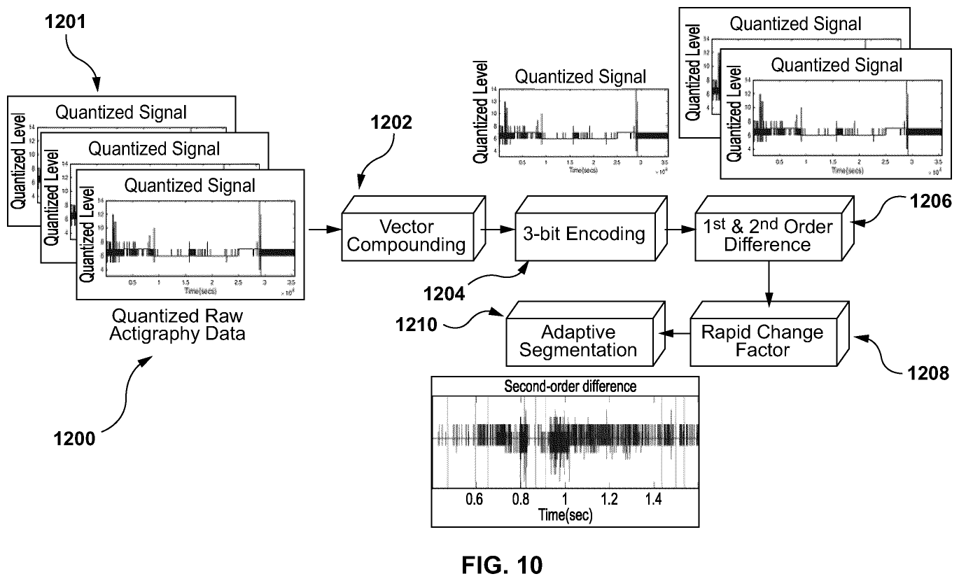

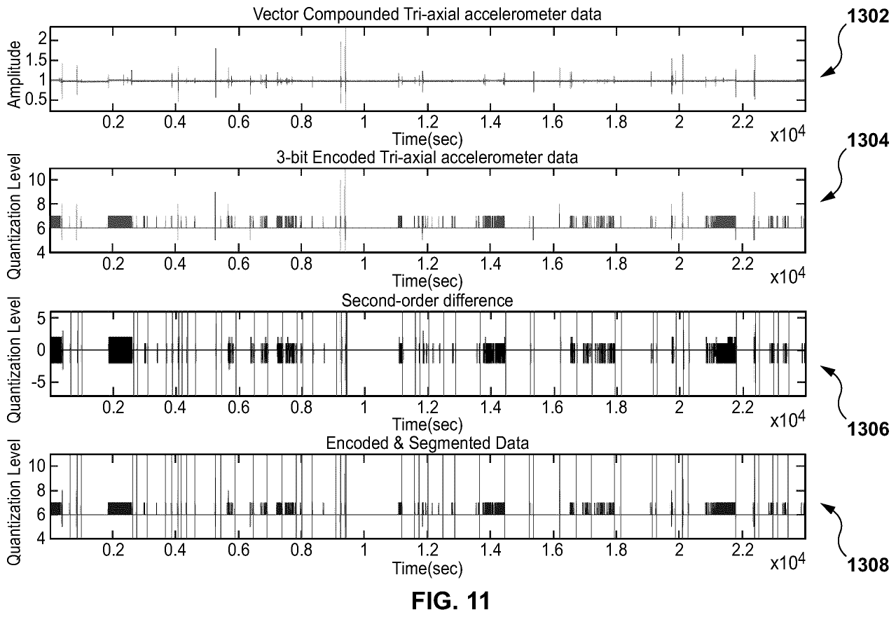

Referring to FIGS. 10 and 11, shown is an overview example schematic diagram of the operation 1200 performed by the wearable smart device 102 of FIG. 1 in accordance with one embodiment, including receiving a quantized raw actigraphy data 1201, performing vector compounding at block 1202 (e.g. by sampling module 104 of FIG. 1) as shown in example output provided in graph 1302, performing 3-bit encoding at block 1204 (e.g. by encoding module 106 of FIG. 1) as shown in example output provided in graph 1304, performing 1^{st} and 2^{nd} order difference at block 1206 as shown in example output provided in graph 1306, rapid change factor computation at block 1208 and adaptive segmentation at block 1210 (e.g. by segmentation module 108) to provide segmented data as shown in example output provided in graph 1308.

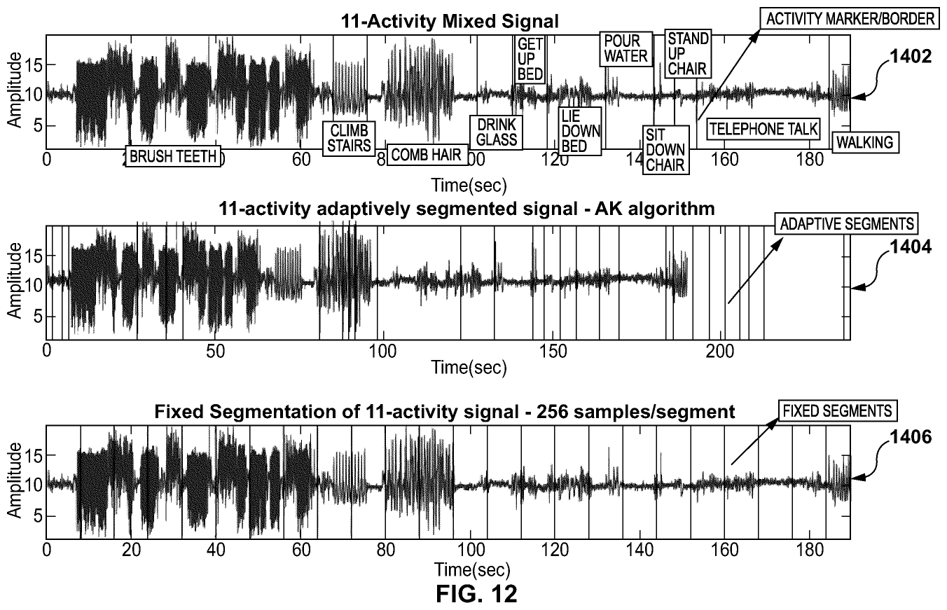

Referring to FIG. 12, shown are example graphs of segmentation based on a mixed activity actigraph signal in graph 1402 (having 11 different activity regions as marked). Graph 1404 illustrates an example segmentation output provided by module 103 of FIG. 1 for providing adaptive segmentation whereas graph 1406 illustrate a technique of fixed segmentation output which does not accurately capture the various activities of graph 1402.

While this specification contains many specifics, these should not be construed as limitations, but rather as descriptions of features specific to particular implementations. Certain features that are described in this specification in the context of separate implementations may also be implemented in combination in a single implementation. Conversely, various features that are described in the context of a single implementation may also be implemented in multiple implementations separately or in any suitable sub-combination. Moreover, although features may be described above as acting in certain combinations and even initially claimed as such, one or more features from a claimed combination may in some cases be excised from the combination, and the claimed combination may be directed to a sub-combination or variation of a sub-combination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems may generally be integrated together in a single software product or packaged into multiple software products.

Various embodiments have been described herein with reference to the accompanying drawings. It will, however, be evident that various modifications and changes may be made thereto, and additional embodiments may be implemented, without departing from the broader scope of the disclosed embodiments as set forth in the claims that follow. Further, other embodiments will be apparent to those skilled in the art from consideration of the specification and practice of one or more embodiments of the present disclosure. It is intended, therefore, that this disclosure and the examples herein be considered as exemplary only, with a true scope and spirit of the disclosed embodiments being indicated by the following listing of exemplary claims.

1. A smart wearable device, the device comprising:
(a) one or more sensors, wherein at least one sensor is an accelerometer configured to acquire an actigraphy signal indicating movement information related to a user;
(b) a memory;
(c) one or more communications interfaces;
(d) a processor coupled to the memory; and
(e) programming residing in a non-transitory computer readable medium, wherein the programming is executable by the computer processor and configured to:
(i) receive input from the accelerometer providing the actigraphy signal;
(ii) encode the actigraphy signal using an m-bit encoder;
(iii) determine a first order difference signal of the encoded signal by subtracting every signal component from a value adjacent to it in the encoded actigraphy signal;
(iv) determine a second order difference signal from the first order difference signal, by performing the operation in step (iii) again;
(v) calculate a rapid change factor RCF using the second order difference signal, the rapid change factor RCF based on a spurious free dynamic range R of the second order difference signal, the step size
$$SS ( SS = \frac{R}{{2}^{m} - 1} )$$
of the encoder such that the rapid change factor is calculated as
$$RCF\_factor = \frac{SS}{m \times {t}_{s}}$$
such that tₛ is the sampling period of the signal;
(vi) automatically define, in real-time, primary segment boundaries on the second order difference signal by:
scanning through the second order difference signal and determining signal samples having avalue greater than the RCF factor, wherein time values of said signal samples greater than the RCF factor define primary segment boundaries;
(vii) extracting frames of the encoded actigraphy signal which are between two consecutive primary segment boundaries, said extracted frames defining a compressed actigraphy signal for subsequent communication via said communication interfaces and discarding outlying regions of the second order difference signal.

2. The device of claim 1, wherein the m-bit encoder is a 3-bit encoder with m=3.

3. The device of claim 1, wherein the spurious free dynamic range is the difference in decibels, between an amplitude of fundamental value of the second order difference signal and an amplitude of largest peak in the second order difference signal.

4. The device of claim 3, wherein the received actigraphy signal is tri-axial and of the form: S=<x,y,z>, and vector compounding is performed on the actigraphy signal prior to said encoding of actigraphy signal by determining the vector compounded signal as: V=\sqrt{x²+y²+_{z}²}, wherein the encoding is performed on the vector compounded signal.

5. The device of claim 1, wherein prior to encoding the actigraphy signal, the processor is further configured to:
normalize the actigraphy signal by removing effects of earth's gravitational effects on the accelerometer, depending upon pre-defined device characteristics and a pre-defined activity type associated with the accelerometer.

6. The device of claim 4, wherein encoding further comprises:
computing a quantization factor as follows:
$$swing = \frac{{2}^{m} - 1}{2} ,$$
where m is a desired quantization level;
encoding the vector compounded actigraphy signal using a floor operation: Q=[V×swing+swing].

7. The device of claim 1 further comprising:
the computer readable medium having stored thereon a database comprising a list of pre-defined activity types and associated list of pre-defined duration of time for each of said pre-defined activity types based on previously defined activities associated with the user;
the programming further comprising an application for execution by the processor and further configured to:
receive an input indicating a current activity type being performed by the user;
determine a first duration of time for said current activity type from said list of pre-defined duration of time, wherein extracting frames of the second order difference signal further comprises extracting only said frames between two consecutive primary segment boundaries having a duration of time proximate to said first duration of time and discarding other remaining frames.

8. The device of claim 1, further comprising:
the computer readable medium having stored thereon a database comprising a list of pre-defined activity types and associated signal characteristics comprising: a list of pre-defined duration of time for each of said pre-defined activity types based on previously defined activities associated with the user;
the programming further comprising an application for execution by the processor and further configured to:
compare said second order difference signal in real-time to said list of signal characteristics to determine a particular activity type for an activity currently being performed by the user;
determine a first duration of time for said particular activity type from said list of pre-defined duration of time, wherein extracting frames of the second order difference signal further comprises extracting only said frames between two consecutive primary segment boundaries having a duration of time proximate to said first duration of time and discarding other remaining frames.

9. The device of claim 1, further comprising a user interface and the programming further comprising: receiving feedback from a user of the device indicating whether the particular activity type was correctly identified and further updating the database of pre-defined activity types and signal characteristics according to said feedback for subsequent definition of time intervals associated with activity types.

10. A computer system comprising a wearable computing device communicating compressed actigraphy data to an external computing device:
the wearable computing device comprising:
a computer readable memory;
an accelerometer sensor;
a processor coupled to the memory;
an application stored on the computer readable memory for execution by the processor for receiving an actigraphy signal related to a user's physical activity from the accelerometer sensor and for compressing the actigraphy signal by determining regions of interest in the actigraphy signal to capture, said compressing performed by:
computing a rapid change factor value indicating a drastic change in movement activity in said actigraphy signal, said rapid change factor computed based on determining a spurious free dynamic range of a second order difference signal of the actigraphy signal and subsequently determining the step size of the actigraphy signal, the step size indicating the interval with which the actigraphy signal instantaneously changes its value from one sample to another;
automatically scanning the second order difference signal to locate samples in the second order difference signal having a value greater than the rapid change factor value, said located samples defining primary segment boundaries;
extracting frames of the second order difference signal between two consecutive primary segment boundaries and discarding outlying regions of the second order difference signal; and
outputting only the extracted frames to the external computing device.

11. The system of claim 10 further comprising:
the computer readable memory having stored thereon a database comprising a list of pre-defined activity types and associated list of pre-defined duration of time for each of said pre-defined activity types based on previously defined activities associated with the user;
the application for execution by the processor further configured to: receive an input indicating a current activity type being performed by the user; determine a first duration of time for said current activity type from said list of pre-defined duration of time, wherein extracting frames of the second order difference signal further comprises extracting only said frames between two consecutive primary segment boundaries having a duration of time proximate to said first duration of time and discarding other remaining frames.

12. A computer program product for dynamically compressing an actigraphy signal at a source device, the computer program product comprising a non-transitory computer-readable medium having computer readable code embodied therein executable by a processor for performing a method for compressing the actigraphy signal, the method comprising:
receiving the actigraphy signal related to a user's physical activity from an accelerometer sensor on the source device and for compressing the actigraphy signal by determining regions of interest in the actigraphy signal to capture, said compressing performed by:
computing a rapid change factor value indicating a drastic change in movement activity in said actigraphy signal, said rapid change factor computed based on determining a spurious free dynamic range of a second order difference signal of the actigraphy signal and subsequently determining the step size of the actigraphy signal, the step size indicating the interval with which the actigraphy signal instantaneously changes its value from one sample to another;
automatically scanning the second order difference signal to locate samples in the second order difference signal having a value greater than the rapid change factor value, said located samples defining primary segment boundaries;
extracting frames of the encoded actigraphy signal between two consecutive primary segment boundaries and discarding outlying regions of the encoded actigraphy signal; and
outputting only the extracted frames representing a compressed actigraphy signal to an external computing device for subsequent processing.

13. A method of determining regions of interest to extract in an actigraphy signal acquired by a wearable smart device, the method executed in a processor of the wearable smart device and comprising the steps of:
a) computing a rapid change factor value indicating a drastic change in movement activity in said actigraphy signal, said rapid change factor computed based on determining a spurious free dynamic range of a second order difference signal of the actigraphy signal and subsequently determining the step size of the actigraphy signal, the step size indicating the interval with which the actigraphy signal instantaneously changes its value from one sample to another;
b) automatically scanning the second order difference signal to locate samples in the second order difference signal having a value greater than the rapid change factor value, said located samples defining primary segment boundaries;
c) extracting frames of the encoded actigraphy signal between two consecutive primary segment boundaries and discarding outlying regions of the second order difference signal; and
d) outputting only the extracted frames representing a compressed actigraphy signal to an external computing device.

14. A computer program product for dynamically compressing an actigraphy signal at a source device, the computer program product comprising a non-transitory computer-readable medium having computer readable code embodied therein executable by a processor for performing a method for compressing the actigraphy signal, the method comprising:
receiving an actigraphy signal indicating movement information related to a user at the source device;
encoding the actigraphy signal using an m-bit encoder;
determining a first order difference signal of the encoded signal by subtracting every signal component from a value adjacent to it in the encoded actigraphy signal;
determining a second order difference signal from the first order difference signal, by performing the operation in step (iii) again;
calculating a rapid change factor RCF using the second order difference signal, the rapid change factor RCF based on a spurious free dynamic range R of the second order difference signal, the step size
$$SS ( SS = \frac{R}{{2}^{m} - 1} )$$
of the encoder such that the rapid change factor is calculatea as
$$RCF\_factor = \frac{SS}{m \times {t}_{s}}$$
such that tₛ is the sampling period of the signal;
automatically defining, in real-time, primary segment boundaries on the second order difference signal by:
scanning through the second order difference signal and determining signal samples having a value greater than the RCF factor, wherein time values of said signal samples greater than the RCF factor define primary segment boundaries;
extracting frames of the encoded actigraphy signal which are between two consecutive primary segment boundaries, said extracted frames defining a compressed actigraphy signal for subsequent communication via said communication interfaces and discarding outlying regions of the second order difference signal.

15. The computer program product of claim 14, wherein the m-bit encoder is a 3-bit encoder with m=3.

16. The computer program product of claim 14, wherein the spurious free dynamic range is the difference in decibels, between an amplitude of fundamental value of the second order difference signal and an amplitude of largest peak in the second order difference signal.

17. The computer program product of claim 14, wherein the received actigraphy signal is tri-axial and of the form: S=<x,y,z>, and the method further comprises vector compounding on the actigraphy signal prior to said encoding of actigraphy signal by determining the vector compounded signal as: V=\sqrt{x²+y²+z²}, wherein the encoding is performed on the vector compounded signal.

18. The computer program product of claim 14, wherein prior to encoding the actigraphy signal, the method further comprises:
normalizing the actigraphy signal by removing effects of earth's gravitational effects on the accelerometer, depending upon pre-defined device characteristics and a pre-defined activity type associated with the accelerometer.

19. The computer program product of claim 18, wherein encoding further comprises:
computing a quantization factor as follows:
$$swing = \frac{{2}^{m} - 1}{2} ,$$
where m is a desired quantization level;
encoding the vector compounded actigraphy signal using a floor operation: Q=[V×swing+swing].

20. The computer program product of claim 14, wherein the method further comprises:
providing a database comprising a list of pre-defined activity types and associated list of pre-defined duration of time for each of said pre-defined activity types based on previously defined activities associated with the user;
and the method further comprising:
receiving an input indicating a current activity type being performed by the user;
determining a first duration of time for said current activity type from said list of pre-defined duration of time, wherein extracting frames of the actigraphy signal further comprises extracting only said frames between two consecutive primary segment boundaries having a duration of time proximate to said first duration of time and discarding other remaining frames.

21. The computer program product of claim 14, further comprising:
providing a database comprising a list of pre-defined activity types and associated signal characteristics comprising: a list of pre-defined duration of time for each of said pre-defined activity types based on previously defined activities associated with the user;
the method further comprising an application for execution by the processor and further configured to:
compare said second order difference signal in real-time to said list of signal characteristics to determine a particular activity type for an activity currently being performed by the user;
determine a first duration of time for said particular activity type from said list of pre-defined duration of time, wherein extracting frames of the actigraphy signal further comprises extracting only said frames between two consecutive primary segment boundaries having a duration of time proximate to said first duration of time and discarding other remaining frames.
